(19) United States
(12) Patent Application Publication
(10) Pub. No.: US 2008/0147021 A1
(43) Pub. Date: Jun. 19, 2008

(54) DRUG DELIVERY DEVICES

(76) Inventor: Dharmendra M. Jani, Fairport, NY (US)

Correspondence Address: Glenn D. Smith, Esq.;Bausch & Lomb Incorporated, One Bausch & Lomb Place, Rochester, NY, 14604, US

(21) Appl. No.: 11/611,503

(22) Filed: Dec. 15, 2006

(51) Int. Cl. A61M 37/00

(52) U.S. Cl. 604/288.01

(57) ABSTRACT

A drug delivery device that is suitable for delivery of a therapeutic agent to limited access regions of the eye is provided. Preferred devices of the invention are minimally invasive, refillable and may be easily fixed to the treatment area.

### BACKGROUND OF THE INVENTION

1. Technical Field

The present invention generally relates to a drug delivery device and method for intraocular delivery of therapeutic agents.

2. Description of Related Art

The delivery of drugs to the eye presents many challenges. For example, the ocular absorption of systemically administered pharmacologic agents can be limited by the blood ocular barrier, namely the tight junctions of the retinal pigment epithelium and vascular endothelial cells. High systemic doses can penetrate this blood ocular barrier in relatively small amounts, but expose the patient to the risk of systemic toxicity. Topical delivery of drugs can result in limited ocular absorption due to the complex hydrophobic/hydrophilic properties of the cornea and sclera. Additionally, topical agents can be mechanically removed by the blink mechanism such that only a limited amount of a single drop may be absorbed. Diffusion of topically administered drugs to the posterior chamber occurs, but often at sub-therapeutic levels. Intravitreal injection of drugs can be an effective means of delivering a drug to the posterior segment in high concentrations. However, these repeated intraocular injections carry the risk of infection, hemorrhage and retinal detachment. Patients may find this procedure somewhat difficult to endure.

Another alternative for drug delivery to the eye is a tacking device. For example, U.S. Pat. No. 4,712,550 discloses a retinal tack for securing a human patient's detached retina to the choroids. Another example is U.S. Pat. No. 5,466,233 (“the '233 patent”) which discloses a tack for intraocular drug delivery. The '233 patent further discloses that the tack consists of a post containing a drug to be administered and having a first end for being positioned within a vitreous region of an eye and a second end which is affixed to an anchoring region having a head extending radially outwardly from the anchoring region such that upon insertion of the anchoring region and post within the eye, the head remains external to the eye and abuts a scleral surface of the eye. The post disclosed in the '233 patent can be an elastomeric material, a solid, non-erodible polymeric matrix having drug particles dispersed therein, or a bio-erodible polymer matrix having drug particles dispersed therein.

Yet another example is U.S. Pat. No. 5,707,643 (“the '643 patent”) which discloses a scleral plug made of a lactic acid copolymer of lactic acid units and glycolic acid units, and containing a drug to be delivered into a vitreous body for treating or preventing diseases of the retina. The '643 patent further discloses that the scleral plug needs to be strong enough not to break or chip by manipulation with a pincette during surgery, and further needs to have properties to release a drug slowly during the desired period of time for treatment and to degrade and be absorbed in the eye tissue afterwards.

A problem associated with the use of a scleral tack formed from a biodegradable material is the effect of the degradation products resulting from the biodegradable material on the tissue in the body, e.g., toxicity levels of the biodegradable material such as lactic and glycolic acid can be delicate to ocular tissues when it comes in contact with the tissue. In addition, fragmentation of the biodegradable tack might release a high dose of drug to the tissues and large fragments into the vitreous body which can impede vision. Accordingly, it would be desirable to provide improved drug delivery devices for delivering a drug to an area of the eye in need of treatment.

### SUMMARY OF THE INVENTION

In accordance with a first embodiment of the present invention, an implantable drug delivery device for intraocular delivery is provided comprising:
(a) a non-deformable, non-degradable, substantially linear shaped body member for housing a polymeric matrix comprising one or more pharmaceutically active agents and being implanted within a patient's eye during use of the device to deliver the one or more pharmaceutically active agents to the patient's eye;
(b) a delivery mechanism for delivery of the one or more pharmaceutically active agents; and
(c) a cap element that remains external to the eye and mates against the outer surface of the patient's eye while the substantially linear shaped body member is inserted into the eye.

In accordance with a second embodiment of the present invention, a method for the treatment of a state, disease, disorder, injury or condition of the eye of a patient is provided which comprises
(a) providing a drug delivery device comprising (i) a non-deformable, non-degradable, substantially linear shaped body member for housing a polymeric matrix comprising one or more pharmaceutically active agents to be delivered; (ii) a delivery mechanism for delivery of the one or more pharmaceutically active agents; and (iii) a cap element that remains external to the eye and mates against the outer surface of the patient's eye while the substantially linear shaped body member is inserted into the eye; and
(b) inserting the device into a patient's eye.

The term “non-deformable” as used herein shall be understood to mean a material that when subjected to the expansive forces of the polymeric matrix housed therein, under use conditions, is rigid enough such that it inhibits the general swelling of the polymeric matrix due to water absorption and does not appreciably expand. It should be understood that localized swelling of the polymeric matrix may take place in areas where the polymeric matrix is in intimate contact with the aqueous environment, e.g., openings of the body member.

The term “treating” or “treatment” of a state, disease, disorder, injury or condition as used herein shall be understood to mean (1) preventing or delaying the appearance of clinical symptoms of the state, disease, disorder, injury or condition developing in a mammal that may be afflicted with or predisposed to the state, disease, disorder, injury or condition but does not yet experience or display clinical or subclinical symptoms of the state, disease, disorder, injury or condition, (2) inhibiting the state, disease, disorder, injury or condition, i.e., arresting or reducing the development of the disease or at least one clinical or subclinical symptom thereof, or (3) relieving the state, disease, disorder, injury or condition, i.e., causing regression of the state, disease, disorder, injury or condition or at least one of its clinical or subclinical symptoms.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein shall be understood to mean providing a therapeutically effective amount of a pharmaceutically active agent to a particular location within a host causing a therapeutically effective concentration of the pharmaceutically active agent at the particular location.

The term “subject” or “patient” or “host” or “mammal” as used herein refers to mammalian animals and humans.

The terms “drug” and “pharmaceutically active agent” shall be used interchangeably herein.

### BRIEF DESCRIPTION OF THE DRAWINGS

FIG. 1 is a perspective view of a drug delivery device of the present invention.

FIG. 2 is a perspective view of an alternative drug delivery device of the present invention.

FIG. 3 is a perspective view of an alternative drug delivery device of the present invention.

FIG. 4 is a perspective view of an alternative drug delivery device of the present invention.

FIG. 5 is a perspective view of a drug delivery device of the present invention.

FIG. 6 (including exploded view FIG. 6A) illustrates a cross-sectional view of an eye having a drug delivery device shown in FIG. 1 positioned therein in accordance with one embodiment of the present invention.

FIG. 7 is a graphical representation depicting the drug release rate over time for a drug loaded non-deformable tack of the present invention.

### DETAILED DESCRIPTION OF THE PREFERRED EMBODIMENTS

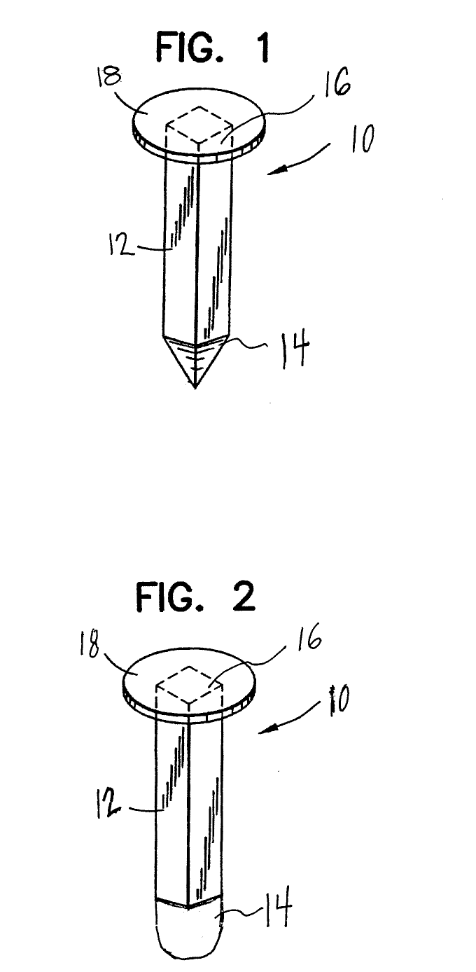

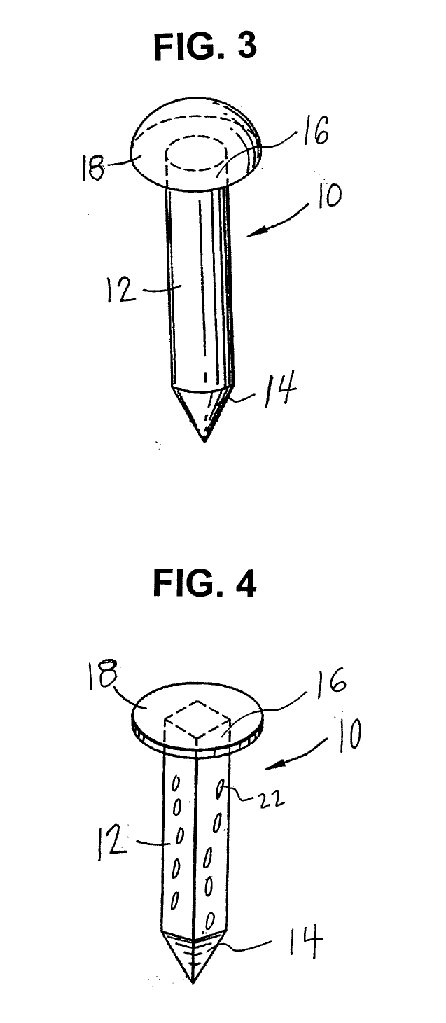

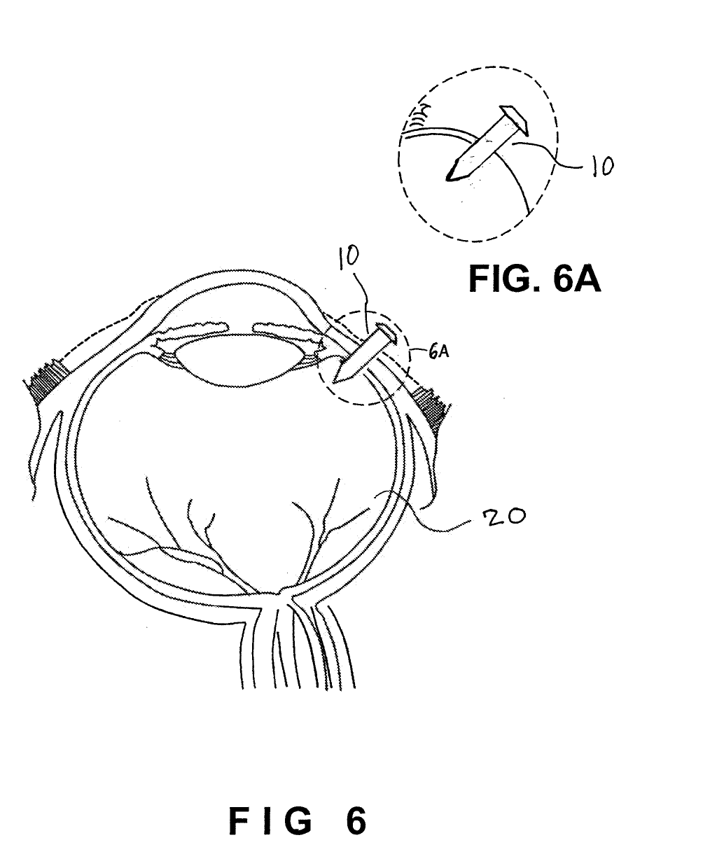

The present invention is directed to a drug delivery device for the treatment of a state, disease, disorder, injury or condition in the eye of a mammal. In one embodiment, the drug delivery device of the present invention is a scleral tack. As shown in FIGS. 1-6, the drug delivery device, generally designated 10, of the present invention includes at least a non-deformable, non-degradable, substantially linear shaped body member 12 and a cap element 18 for securing device 10 within the eye 20.

Non-deformable, non-degradable, substantially linear shaped body member 12 will have a substantially linear shape and a distal end 14 and a proximal end 16. In general, substantially linear shaped body member 12 can be conical in shape at distal end 14 as shown in FIGS. 1, 3, 4 and 5 or capsule-shaped as shown in FIG. 2. However, substantially linear shaped body member 12 may have any configuration or shape at distal end 14. In one embodiment, a shape of the drug delivery device is a nail-like shape comprising a head portion, which prevents the plug from dropping into the vitreous body, and a shaft portion, which is inserted into a scleral incision. In particular, it is preferable that distal end 14 of substantially linear shaped body member 12 be pointed, i.e., it is an acute-angled shape such as pyramidal or conical to prevent disease complication, which may be caused when the device is inserted into the eye.

In general, useful materials in fabricating substantially linear shaped body member 12 are not particularly limited, provided these materials are biocompatible, non-deformable and non-degradable and/or approved for use by United States Food and Drug Administration (“FDA”) for administration for intraocular use in humans or, in keeping with established regulatory criteria and practice, is susceptible to approval by the FDA for for intraocular use in humans. Suitable non-deformable, non-degradable materials for forming substantially linear shaped body member 12 include materials will have a Youngs Modulus of at least about 1 GPa. In another embodiment, suitable non-deformable, non-degradable materials for forming substantially linear shaped body member 12 include materials will have a Youngs Modulus of at least about 100 GPa. Representative examples of such materials include, but are not limited to, steels, e.g., stainless steels such as Class VI stainless steels, e.g., 316L stainless steel grade and the like; metal alloys, e.g., cobalt-chromium-molybdenum alloy and the like; titanium-containing material, e.g., 6Al-4V Grade 5, 6Al4V-ELI Grade 23 and the like; ceramics, e.g., hydroxyl apatite and the like, ultra-high molecular weight polyethylene (UHMWPE), polymethylmethacrylate (PMMA), polyether ether ketone (PEEK) and the like and mixtures thereof. In one embodiment, the non-deformable, non-degradable material is a non-elastomeric material. In another embodiment, the non-deformable, non-degradable material is a non-polymeric material. Methods for making body member 12 are within the purview of one skilled in the art, e.g., micromachining techniques for preparing surgical implants.

As one skilled in the art will readily appreciate, if distal end 14 of substantially linear shaped body member 12 is conical in shape and used to pierce the eye during insertion, at least the distal end 14 can be fabricated of a rigid, non-pliable material suitable for piercing the eye and may be different from the material used in forming substantially linear shaped body member 12 and cap element 18, as described hereinbelow. Such materials are well known in the art and may include, for example, polyimide and the like.

If desired, substantially linear shaped body member 12 can have an anti-microbial or anti-fibrotic coating thereon. Suitable materials for forming the anti-microbial or anti-fibrotic coating are not particularly limiting and are well known in the art. Such materials are typically designed to selectively promote or deter cell activities such as attachment, activation, proliferation or differentiation of endogenous cells, sporogenic and non-sporogenic fungi and eukaryotic and prokaryotic microorganisms, e.g., gram-negative and gram-positive bacteria. The coating can be applied to body member 12 by techniques known in the art, e.g., spraying, dip coating and the like. If desired, different coatings may be applied on various sections of body member 12 to achieve a desired result.

Generally, the rate of release of the pharmaceutically active agents can also be controlled by manipulating the hydrophobic/hydrophilic balance of the polymeric matrix containing the one or more pharmaceutically active agents to achieve the desired rate of drug release, such that the properties of the drug delivery systems, e.g., water content, modulus and glass transition temperature (T_{g}), can be controlled thereby having a pronounced impact on the release characteristics of the one or more pharmaceutically active agents entrapped in the copolymer. For example, in the case of the pharmaceutically active agent fluocinolone acetonide, a relatively hydrophobic drug, it is believed that the release rate can be changed significantly with respect to the water content of the drug delivery system, e.g., by controlling the balance of the hydrophobic and hydrophilic monomers in the copolymer, a suitable water content of the system can be achieved which, in turn, will control the release of the drug. Accordingly, the desired rate of drug release may be determined based on, for example, the drug to be delivered, the location of delivery, the copolymer used in making the drug delivery system, the purpose of delivery and/or the therapeutic requirements of the individual patient as discussed above.

Substantially linear shaped body member 12 possesses a hollow region therein for accommodating the polymeric matrix containing the one or more pharmaceutically active agents. In one embodiment, body member 12 may contain more than one polymeric matrix therein such that body member 12 contains compartments containing each polymeric matrix. For example, in one embodiment, body member 12 can contain one polymeric matrix formed from a silicone/poly(methyl methacrylate) copolymer containing one or more pharmaceutically active agents and a second polymeric matrix formed from a poly(2-hydroxyethyl methacrylate containing a drug to prevent transduction of bacteria and fibroblasts.

A suitable polymer/drug matrix for use in the hollow portion of substantially linear shaped body member 12 can be a biocompatible homo- or co-polymer, which can be a biodegradable homo- or co-polymer or a non-biodegradable homo- or co-polymer. Suitable biodegradable polymers for use herein include, but are not limited to, poly(lactides), poly(glycolides), poly(lactide-co-glycolides), poly(lactic acids), poly(glycolic acids), poly(lactic acid-co-glycolic acids), polycaprolactone, polycarbonates, polyesteramides, polyanhydrides, poly(amino acids), polyorthoesters, polyacetals, polycyanoacrylates, polyetheresters, poly(dioxanones), poly(alkylene alkylates), copolymers of polyethylene glycol and polyorthoester, biodegradable polyurethanes, and their blends and copolymers thereof. In one embodiment, a polymeric matrix can be formed from a polylactic-co-glycolic acid (PLGA) containing polymers, for example, PLGA in a ratio of 50/50, 65/35 or 75/25, or copolymers thereof, e.g., 50/50 DL-PLGA, 75/25 DL-PLGA, 50/50 L-PLGA, etc. Methods for making such a polymeric matrix is known in the art, see, e.g., U.S. Patent Application Publication Number 2004/0253293 and 2005/0031669. The one or more pharmaceutically active agents can be combined with the polymeric matrix either during polymerization or subsequent to polymerization by techniques known in the art, e.g., thermal polymerization, solvent entrapment, and the like.

Suitable non-biodegradable polymers for use herein can be any naturally occurring or synthetic material that is biologically compatible with body fluids and eye tissues and essentially insoluble in body fluids which the material will come in contact. Such materials include, but are not limited to, glass, metal, ceramics, polyvinyl acetate, cross-linked polyvinyl alcohol, cross-linked polyvinyl butyrate, ethylene ethylacrylate copolymer, polyethyl hexylacrylate, polyvinyl chloride, polyvinyl acetals, plasiticized ethylene vinylacetate copolymer, polyvinyl alcohol, polyvinyl acetate, ethylene vinylchloride copolymer, polyvinyl esters, polyvinylbutyrate, polyvinylformal, polyamides, polymethylmethacrylate, polybutylmethacrylate, plasticized polyvinyl chloride, plasticized nylon, plasticized soft nylon, plasticized polyethylene terephthalate, natural rubber, polyisoprene, polyisobutylene, polybutadiene, polyethylene, polytetrafluoroethylene, polyvinylidene chloride, polyacrylonitrile, cross-linked polyvinylpyrrolidone, polytrifluorochloroethylene, chlorinated polyethylene, poly(1,4′-isopropylidene diphenylene carbonate), vinylidene chloride, acrylonitrile copolymer, vinyl chloride-diethyl fumerate copolymer, butadiene/styrene copolymers, silicone rubbers, especially the medical grade polydimethylsiloxanes, ethylene-propylene rubber, silicone-carbonate copolymers, vinylidene chloride-vinyl chloride copolymer, vinyl chloride-acrylonitrile copolymer, vinylidene chloride-acrylonitride copolymer and the like.

In another embodiment, the polymeric matrix containing the one or more pharmaceutically active agents can be prepared by reacting one or more acrylate ester and/or methacrylate ester-containing monomers with one or more acrylamido-containing monomers optionally in the presence of one or more crosslinking agents. The resulting copolymers can be in random or block sequences.

Suitable acrylate ester and/or methacrylate ester-containing monomers may be represented by the general formula:

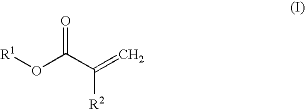

wherein R¹ may be a C₁-C₁₈ alkyl, C₃-C₁₈ cycloalkyl, C₃-C₁₈ cycloalkylalkyl, C₃-C₁₈ cycloalkenyl, C₅-C₃₀ aryl, C₅-C₃₀ arylalkyl, C₁-C₁₈ alkyl siloxysilane, C₁-C₁₈ alkyl siloxane, ether or polyether-containing groups, substituted or unsubstituted, linear or branched, and R² is H or CH₃.

Representative examples of alkyl groups for use herein include, by way of example, a straight or branched hydrocarbon chain radical containing carbon and hydrogen atoms of from 1 to about 18 carbon atoms with or without unsaturation, to the rest of the molecule, e.g., methyl, ethyl, n-propyl, 1-methylethyl (isopropyl), n-butyl, n-pentyl, etc., and the like.

Representative examples of cycloalkyl groups for use herein include, by way of example, a substituted or unsubstituted non-aromatic mono or multicyclic ring system of about 3 to about 18 carbon atoms such as, for example, cyclopropyl, cyclobutyl, cyclopentyl, cyclohexyl, perhydronapththyl, adamantyl and norbornyl groups bridged cyclic group or sprirobicyclic groups, e.g., sprio-(4,4)-non-2-yl and the like, optionally containing one or more heteroatoms, e.g., O and N, and the like.

Representative examples of cycloalkylalkyl groups for use herein include, by way of example, a substituted or unsubstituted cyclic ring-containing radical containing from about 3 to about 18 carbon atoms directly attached to the alkyl group as defined above which is then attached to the main structure of the monomer (via the oxygen atom) at any carbon atom from the alkyl group that results in the creation of a stable structure such as, for example, cyclopropylmethyl, cyclobutylethyl, cyclopentylethyl and the like, wherein the cyclic ring can optionally contain one or more heteroatoms, e.g., O and N, and the like.

Representative examples of cycloalkenyl groups for use herein include, by way of example, a substituted or unsubstituted cyclic ring-containing radical containing from about 3 to about 18 carbon atoms with at least one carbon-carbon double bond such as, for example, cyclopropenyl, cyclobutenyl, cyclopentenyl and the like, wherein the cyclic ring can optionally contain one or more heteroatoms, e.g., O and N, and the like.

Representative examples of aryl groups for use herein include, by way of example, a substituted or unsubstituted monoaromatic or polyaromatic radical containing from about 5 to about 25 carbon atoms such as, for example, phenyl, naphthyl, tetrahydronapthyl, indanyl, biphenyl and the like, optionally containing one or more heteroatoms, e.g., O and N, and the like.

Representative examples of arylalkyl groups for use herein include, by way of example, a substituted or unsubstituted aryl group as defined above directly attached to an alkyl group as defined above which is then attached to the main structure of the monomer (via the oxygen atom) at any carbon atom from the alkyl group that results in the creation of a stable structure, e.g., —CH₂C₆H₅, —C₂H₅C₆H₅ and the like, wherein the aryl group can optionally contain one or more heteroatoms, e.g., O and N, and the like.

Representative examples of alkyl siloxysilane groups for use herein include, by way of example, a siloxysilane group directly attached to an alkyl group as defined above which is then attached to the main structure of the monomer (via the oxygen atom) at any carbon atom from the alkyl group that results in the creation of a stable structure, e.g., —(CH₂)ₕ siloxysilane such as one represented by the following structure:

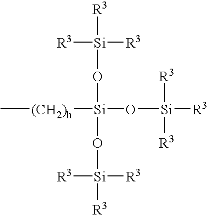

wherein h is 1 to 18 and each R³ independently denotes an lower alkyl radical, phenyl radical or a group represented by

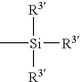

wherein each R^{3′} independently denotes a lower alkyl or aryl radical as defined above. Representative examples of such acrylate ester and/or methacrylate ester-containing monomers include 3-methacryloyloxypropyltris(trimethylsiloxy)silane or tris(trimethylsiloxy)silylpropyl methacrylate, sometimes referred to as TRIS and tris(trimethylsiloxy)silylpropyl vinyl carbamate, sometimes referred to as TRIS-VC and the like and are commercially available from such sources as Gelest, Inc. (Morrisville, Pa.) and can be prepared by methods well known in the art.

Representative examples of alkyl siloxane groups for use herein include, by way of example, a siloxane group directly attached to an alkyl group as defined above which is then attached to the main structure of the monomer (via the oxygen atom) at any carbon atom from the alkyl group that results in the creation of a stable structure, e.g.,—(CH₂)ₓ siloxane such as one represented by the following structure:

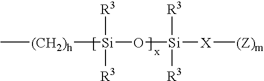

wherein x is an integer from 0 to about 300; h is an integer from 1 to 18, m is an integer from 1 to about 6, each R³ is independently hydrogen, or a lower alkyl or aryl radical as defined above; X is a bond, straight or branched C₁-C₃₀ alkyl group, a C₁-C₃₀ fluoroalkyl group, a substituted or unsubstituted C₅-C₃₀ arylalkyl group, a substituted or unsubstituted C₁-C₃₀ alkoxy group, an ether or polyether containing group, sulfide, or amino-containing group and Z is a polymerizable ethylenically unsaturated organic radical, e.g., (meth)acrylate-containing radicals, (meth)acrylamide-containing radicals, vinylcarbonate-containing radicals, vinylcarbamate-containing radicals, styrene-containing radicals and the like. A representative example of such an acrylate ester and/or methacrylate ester-containing monomer includes α,ω-methacrylate end capped polydimethyl(siloxanes) and the like and are commercially available from such sources as Gelest, Inc. (Morrisville, Pa.) and can be prepared by methods well known in the art.

Representative examples of ether or polyether-containing groups for use herein include, by way of example, an alkyl ether, cycloalkyl ether, cycloalkylalkyl ether, cycloalkenyl ether, aryl ether, arylalkyl ether wherein the alkyl, cycloalkyl, cycloalkylalkyl, cycloalkenyl, aryl, and arylalkyl groups are defined above, e.g., alkylene oxides, poly(alkylene oxide)s such as ethylene oxide, propylene oxide, butylene oxide, poly(ethylene oxide)s, poly(ethylene glycol)s, poly(propylene oxide)s, poly(butylene oxide)s and mixtures thereof, an ether or polyether group of the general formula —R⁴OR^{4′}, wherein R⁴ is a bond, an alkyl, cycloalkyl or aryl group as defined above and R^{4′} is an alkyl, cycloalkyl or aryl group as defined above, e.g., —CH₂CH₂OC₆H₅ and —CH₂CH₂OC₂H₅, and the like.

The substituents in the ‘substituted alkyl’, ‘substituted cycloalkyl’, ‘substituted cycloalkylalkyl’, ‘substituted cycloalkenyl’, ‘substituted arylalkyl’ and ‘substituted aryl’ may be the same or different with one or more selected from the group such as hydrogen, halogen (e.g., fluorine), substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkenyl, substituted or unsubstituted aryl, substituted or unsubstituted heteroaryl, substituted heterocyclylalkyl ring, substituted or unsubstituted heteroarylalkyl, substituted or unsubstituted heterocyclic ring.

In one embodiment, useful acrylate ester or methacrylate ester-containing monomers include, but are not limited to, a linear or branched, substituted or unsubstituted, C₁ to C₁₈ alkyl acrylate, a linear or branched, substituted or unsubstituted, C₁ to C₁₈ alkyl methacrylate, a substituted or unsubstituted C₃ to C₁₈ cycloalkyl acrylate, a substituted or unsubstituted C₃ to C₁₈ cycloalkyl methacrylate, a substituted or unsubstituted C₆ to C₂₅ aryl or alkaryl acrylate, a substituted or unsubstituted C₆ to C₂₅ aryl or alkaryl methacrylate, an ethoxylated acrylate, an ethoxylated methacrylate, partially fluorinated acrylates, partially fluorinated methacrylates and the like and mixtures thereof. In another embodiment, the acrylate ester and/or methacrylate ester-containing monomers are hydrophobic monomers.

Representative examples of acrylate ester-containing monomers for use herein include, but are not limited to, methyl acrylate, ethyl acrylate, propyl acrylate, isopropyl acrylate, n-butyl acrylate, iso-butyl acrylate, t-butyl acrylate, n-hexyl acrylate, 2-ethylbutyl acrylate, 2-ethylhexyl acrylate, cyclopropyl acrylate, cyclobutyl acrylate, cyclohexyl acrylate, benzyl acrylate, 2-phenoxyethyl acrylate, phenyl acrylate, 2-phenylethyl acrylate, 3-phenylpropyl acrylate, 3-phenoxypropyl acrylate, 4-phenylbutyl acrylate, 4-phenoxybutyl acrylate, 4-methylphenyl acrylate, 4-methylbenzyl acrylate, 2-2-methylphenylethyl acrylate, 2-3-methylphenylethyl acrylate, 2-methylphenylethyl acrylate and the like and mixtures thereof.

Representative examples of methacrylate ester-containing monomers for use herein include, but are not limited to, methyl methacrylate, ethyl methacrylate, propyl methacrylate, isopropyl methacrylate, n-butyl methacrylate, iso-butyl methacrylate, t-butyl methacrylate, n-hexyl methacrylate, 2-ethylbutyl methacrylate, 2-ethylhexyl methacrylate, cyclopropyl methacrylate, cyclobutyl methacrylate, cyclohexyl methacrylate, benzyl methacrylate, 2-phenoxyethyl methacrylate, phenyl methacrylate, 2-phenylethyl methacrylate, 3-phenylpropyl methacrylate, 3-phenoxypropyl methacrylate, 4-phenylbutyl methacrylate, 4-phenoxybutyl methacrylate, 4-methylphenyl methacrylate, 4-methylbenzyl methacrylate, 2-2-methylphenylethyl methacrylate, 2-3-methylphenylethyl methacrylate, 2-4-methylphenylethyl methacrylate and the like and mixtures thereof.

Suitable acrylamido-containing monomers may be represented by the general formulae II and III

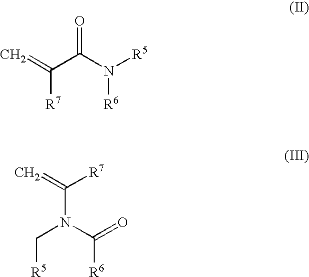

wherein R⁵ and R⁶ are independently hydrogen, a C₁-C₁₈ alkyl, C₃-C₁₈ cycloalkyl, C₃-C₁₈ cycloalkylalkyl, C₃-C₁₈ cycloalkenyl, C₅-C₃₀ aryl, C₅-C₃₀ arylalkyl, C₁-C₁₈ alkyl siloxysilane or C₁-C₁₈ alkyl siloxane, substituted or unsubstituted, linear or branched, as defined above or R⁵ and R⁶ together with the nitrogen atom to which they are bonded are joined together to form a heterocyclic group and R⁷ is H or CH₃.

Representative examples of acrylamido-containing monomers include, but are not limited to, acrylamide, N-methylacrylamide, N-ethylacrylamide, N-propylacrylamide, N-isopropylacrylamide, N-butylacrylamide, N,N-dimethylacrylamide, N,N-diethylacrylamide, N,N-dipropylacrylamide, N,N-dibutylacrylamide, N,N-methylethylacrylamide, N,N-methylpropylacrylamide, N,N-ethylpropylacrylamide, N,N-ethylbutylacrylamide, N,N-propylbutylacrylamide, N-cyclopropylacrylamide, N-cyclobutylacrylamide, N-vinylpyrrolidone and the like and mixtures thereof. In one embodiment, the acrylamido-containing monomers are hydrophilic monomers.

Generally, in one embodiment the acrylate ester and/or methacrylate ester-containing monomer(s) can be added to a reaction mixture in an amount ranging from about 10% w/w to about 80% w/w and preferably from about 20% w/w to about 50% w/w and the acrylamido-containing monomer(s) can be added to the reaction mixture in an amount ranging from about 90% w/w to about 10% w/w and preferably from about 80% w/w to about 30% w/w.

The polymers for use in forming the polymeric matrix can be crosslinked with one or more crosslinking agents. Preferably, the crosslinking agent is one that is copolymerized with the reactive monomers. Suitable crosslinking agents include, but are not limited to, any di- or multi-functional crosslinking agent and the like and mixtures thereof. Representative examples of such crosslinkers include, but are not limited to, tripropylene glycerol diacrylate, ethylene glycol dimethacrylate, tetraethylene glycol dimethacrylate, poly(ethylene glycol diacrylate) (PEG400 or PEG600), methylene bis acrylamide and the like and mixtures thereof. If used, the crosslinking agent is used in an effective amount, by which is meant an amount that is sufficient to cause crosslinking of the monomeric mixture resulting in a copolymer capable of entrapping the one or more pharmaceutically active agents to produce the desired drug delivery system. The amount of the crosslinking agent will ordinarily range from about 0.05% w/w to about 20% w/w and preferably from about 0.1% w/w to about 10% w/w.

In general, the copolymerization reaction can be conducted neat, that is, the one or more monomers, e.g., an acrylate ester and/or methacrylate ester-containing monomer(s) and acrylamido-containing monomer(s), and optional crosslinking agent(s) are combined in the desired ratio, and then exposed to, for example, ultraviolet (UV) light or electron beams in the presence of one or more photoinitiator(s) or at a suitable temperature, for a time period sufficient to form the copolymer. Suitable reaction times will ordinarily range from about 1 minute to about 24 hours and preferably from about 1 hour to about 4 hours.

The use of UV or visible light in combination with photoinitiators is well known in the art and is particularly suitable for formation of the copolymer. Numerous photoinitiators of the type in question here are commercial products. Photoinitiators enhance the rapidity of the curing process when the photocurable compositions as a whole are exposed to, for example, ultraviolet radiation. Suitable photoinitiators which are useful for polymerizing the polymerizable mixture of monomers can be commercially available photoinitiators. They are generally compounds which are capable of initiating the radical reaction of olefinically unsaturated double bonds on exposure to light with a wavelength of, for example, about 260 to about 480 nm.

Examples of suitable photoinitiators for use herein include, but are not limited to, one or more photoinitiators commercially available under the “IRGACURE”, “DAROCUR” and “SPEEDCURE” trade names (manufactures by Ciba Specialty Chemicals, also obtainable under a different name from BASF, Fratelli Lamberti and Kawaguchi), e.g., “IRGACURE” 184 (1-hydroxycyclohexyl phenyl ketone), 907 (2-methyl-1-[4-(methylthio)phenyl]-2-morpholino propan-1-one), 369 (2-benzyl-2-N,N-dimethylamino-1-(4-morpholinophenyl)-1-butanone), 500 (the combination of 1-hydroxy cyclohexyl phenyl ketone and benzophenone), 651 (2,2-dimethoxy-2-phenyl acetophenone), 1700 (the combination of bis(2,6-dimethoxybenzoyl-2,4,4-trimethyl pentyl)phosphine oxide and 2-hydroxy-2-methyl-1-phenyl-propan-1-one), and 819 [bis(2,4,6-trimethyl benzoyl)phenyl phosphine oxide] and “DAROCUR” 1173 (2-hydroxy-2-methyl-1-phenyl-1-propan-1-one) and 4265 (the combination of 2,4,6-trimethylbenzoyldiphenyl-phosphine oxide and 2-hydroxy-2-methyl-1-phenyl-propan-1-one); and the like and mixtures thereof. Other suitable photoimtiators for use herein include, but are not limited to, alkyl pyruvates, such as methyl, ethyl, propyl, and butyl pyruvates, and aryl pyruvates, such as phenyl, benzyl, and appropriately substituted derivatives thereof. Generally, the amount of photoinitiator can range from about 0.05% w/w to about 5% w/w and preferably from about 0.1% w/w to about 1% w/w.

Copolymerization of the monomeric mixture and optional crosslinking agent(s) can be carried out in any known manner. The important factors are intimate contact of the reactive monomers in, for example, the presence of the photoinitiator(s). The components in the reaction mixture can also be added continuously to a stirred reactor or can take place in a tubular reactor in which the components can be added at one or more points along the tube.

In an alternative embodiment, the process may include at least polymerizing the monomeric mixture in the presence of one or more pharmaceutically active agents under polymerization conditions as discussed above such that the pharmaceutically active agent(s) is entrapped in the polymerization product. In this embodiment, it is particularly advantageous to carry out the polymerization process by exposing the monomeric mixture and pharmaceutically active agent(s) to UV or visible light in the presence of one or more photoinitiator(s). As one skilled in the art will readily appreciate, the resulting polymerization product may have some pharmaceutically active agent(s) which is covalently bound to the polymerization product as well as some free starting monomer(s). If desired, these reactants can be removed as discussed hereinbelow.

Generally, pharmaceutically active agents or drugs useful in the drug delivery device of the present invention can be any compound, composition of matter, or mixtures thereof that can be delivered from the device to produce a beneficial and useful result to the eye, especially an agent effective in obtaining a desired local or systemic physiological or pharmacological effect. Examples of such agents include, but are not limited to, anesthetics and pain killing agents such as lidocaine and related compounds, benzodiazepam and related compounds and the like; anti-cancer agents such as 5-fluorouracil, adriamycin and related compounds and the like; anti-fungal agents such as fluconazole and related compounds and the like; anti-viral agents such as trisodium phosphomonoformate, trifluorothymidine, acyclovir, ganciclovir, DDI, AZT and the like; cell transport/mobility impending agents such as colchicine, vincristine, cytochalasin B and related compounds and the like; antiglaucoma drugs such as beta-blockers, e.g., timolol, betaxolol, atenalol, and the like; antihypertensives; decongestants such as phenylephrine, naphazoline, tetrahydrazoline and the like; immunological response modifiers such as muramyl dipeptide and related compounds and the like; peptides and proteins such as cyclosporin, insulin, growth hormones, insulin related growth factor, heat shock proteins and related compounds and the like; steroidal compounds such as dexamethasone, prednisolone and related compounds and the like; low solubility steroids such as fluocinolone acetonide and related compounds and the like; carbonic anhydrase inhibitors; diagnostic agents; antiapoptosis agents; gene therapy agents; sequestering agents; reductants such as glutathione and the like; antipermeability agents; antisense compounds; antiproliferative agents; antibody conjugates; antidepressants; bloodflow enhancers; antiasthmatic drugs; antiparasiticagents; non-steroidal anti inflammatory agents such as ibuprofen and the like; nutrients and vitamins: enzyme inhibitors: antioxidants; anticataract drugs; aldose reductase inhibitors; cytoprotectants; cytokines, cytokine inhibitors, and cytokin protectants; uv blockers; mast cell stabilizers; anti neovascular agents such as antiangiogenic agents, e.g., matrix metalloprotease inhibitors and the like.

Representative examples of additional pharmaceutically active agent for use herein include, but are not limited to, neuroprotectants such as nimodipine and related compounds and the like; antibiotics such as tetracycline, chlortetracycline, bacitracin, neomycin, polymyxin, gramicidin, oxytetracycline, chloramphenicol, gentamycin, erythromycin and the like; anti-infectives; antibacterials such as sulfonamides, sulfacetamide, sulfamethizole, sulfisoxazole; nitrofurazone, sodium propionate and the like; antiallergenics such as antazoline, methapyriline, chlorpheniramine, pyrilamine, prophenpyridamine and the like; anti-inflammatories such as hydrocortisone, hydrocortisone acetate, dexamethasone 21-phosphate, fluocinolone, medrysone, methylprednisolone, prednisolone 21-phosphate, prednisolone acetate, fluoromethalone, betamethasone, triminolone and the like; miotics; anti-cholinesterase such as pilocarpine, eserine salicylate, carbachol, di-isopropyl fluorophosphate, phospholine iodine, demecarium bromide and the like; miotic agents; mydriatics such as atropine sulfate, cyclopentolate, homatropine, scopolamine, tropicamide, eucatropine, hydroxyamphetamine and the like; sympathomimetics such as epinephrine and the like; and prodrugs such as, for example, those described in Design of Prodrugs, edited by Hans Bundgaard, Elsevier Scientific Publishing Co., Amsterdam, 1985. In addition to the foregoing agents, other agents suitable for treating, managing, or diagnosing conditions in a mammalian organism may be entrapped in the copolymer and administered using the drug delivery systems of the current invention. Once again, reference may be made to any standard pharmaceutical textbook such as, for example, Remington's Pharmaceutical Sciences for pharmaceutically active agents.

Any pharmaceutically acceptable form of the foregoing pharmaceutically active agent may be employed in the practice of the present invention, e.g., the free base; free acid; pharmaceutically acceptable salts, esters or amides thereof, e.g., acid additions salts such as the hydrochloride, hydrobromide, sulfate, bisulfate, acetate, oxalate, valerate, oleate, palmitate, stearate, laurate, borate, benzoate, lactate, phosphate, tosylate, mesylate, citrate, maleate, fumarate, succinate, tartrate, ascorbate, glucoheptonate, lactobionate, and lauryl sulfate salts and the like; alkali or alkaline earth metal salts such as the sodium, calcium, potassium and magnesium salts and the like; hydrates; enantiomers; isomers; stereoisomers; diastereoisomers; tautomers; polymorphs, mixtures thereof, prodrugs thereof or racemates or racemic mixtures thereof.

Actual dosage levels of the pharmaceutically active agent(s) in the drug delivery systems of the present invention may be varied to obtain an amount of the pharmaceutically active agent(s) that is effective to obtain a desired therapeutic response for a particular system and method of administration. The selected dosage level therefore depends upon such factors as, for example, the desired therapeutic effect, the route of administration, the desired duration of treatment, and other factors. The total daily dose of the pharmaceutically active agent(s) administered to a host in single or divided doses can vary widely depending upon a variety of factors including, for example, the body weight, general health, sex, diet, time and route of administration, rates of absorption and excretion, combination with other drugs, the severity of the particular condition being treated, etc. Generally, the amounts of pharmaceutically active agent(s) present in the drug delivery systems of the present invention can range from about 1% w/w to about 60% w/w and preferably from about 5% w/w to about 50% w/w.

The polymeric matrix may be manufactured in any suitable form, shape, e.g., circular, rectangular, tubular, and square shapes, or size as long as the polymeric matrix is sized and configured to be accommodated within substantially linear shaped body member 12. Methods of forming the polymeric matrix include, but are not limited to, cast molding, injection/compression molding, extrusion, and other methods known to those skilled in the art.

As further shown in FIGS. 1-6, cap element 18 is located at proximal end 16 of substantially linear shaped body member 12 to assist in stabilizing device 10 once implanted in eye 20. The overall size and shape of cap element 18 is not particularly limited provided that irritation to the eye is limited. For example, while cap element 18 is shown circular in shape, cap element 18 may be of any shape, for example, circular, rectangular, triangular, etc. However, to minimize irritation to the eye, cap element 18 preferably has rounded edges. Further, cap element 18 is designed such that it remains outside the eye and, as such, cap element 18 is sized so that it will not pass into the eye through the opening in the eye through which the device is inserted. The cap element 18 may further be designed such that it can be easily sutured or otherwise secured to the surface surrounding the opening in the eye and may, for example, contain a plurality of holes (not shown) through which sutures may pass. Preferably, drug delivery device 10 is inserted into the eye through an incision until cap element 18 abuts the incision. If desired, cap element 18 may then be sutured to the eye, using one or more holes in the cap element 18, to further stabilize and prevent the device from moving once it is implanted in its desired location. In one embodiment, cap element 18 can be a diffusion limiting cap.

Suitable materials for fabricating cap element 18 are not particularly limited, provided these materials are biocompatible and preferably insoluble in the body fluids and tissues that the device comes into contact with. In one embodiment, cap element 18 can be fabricated of a material that does not cause irritation to the portion of the eye that it contacts. Useful materials are pliable and may include, but are not limited to, various polymers such as, for example, silicone elastomers and rubbers, polyolefins, polyurethanes, acrylates, polycarbonates, polyamides, polyimides, polyesters, polysulfones and the like and mixtures thereof.

If desired, cap element 18 can have a port in fluid communication with body member 12 to allow for filling and refilling of the device after the device has been implanted in the eye to maintain an ongoing, controlled delivery of the one or more pharmaceutically active agents to the target site. In one embodiment, cap element 18 can be removed and the drug loaded polymeric matrix can be reinserted into substantially linear shaped body member 12.

In one embodiment, the delivery mechanism comprises one or more exit apertures located at the distal end of the body member 12. In another embodiment, the delivery mechanism comprises one or more openings 22 along body member 12 as generally depicted in FIG. 4. Openings 22 can be of any shape and is not particularly limiting. The number and size of the openings can be varied and depends on such factors as the desired rate of release of the drug, the material(s) used in forming the polymeric matrix containing the drug as described hereinabove, the amount of drug, the condition being treated, etc.

In another embodiment, the delivery mechanism comprises the material forming body member 12. For example, the material forming body member 12 may be a material that is permeable or semi-permeable to the substance to be delivered and is a non-perforated device. Representative examples of such materials include, ceramics, bioglass and the like and are within the purview of one skilled in the art.

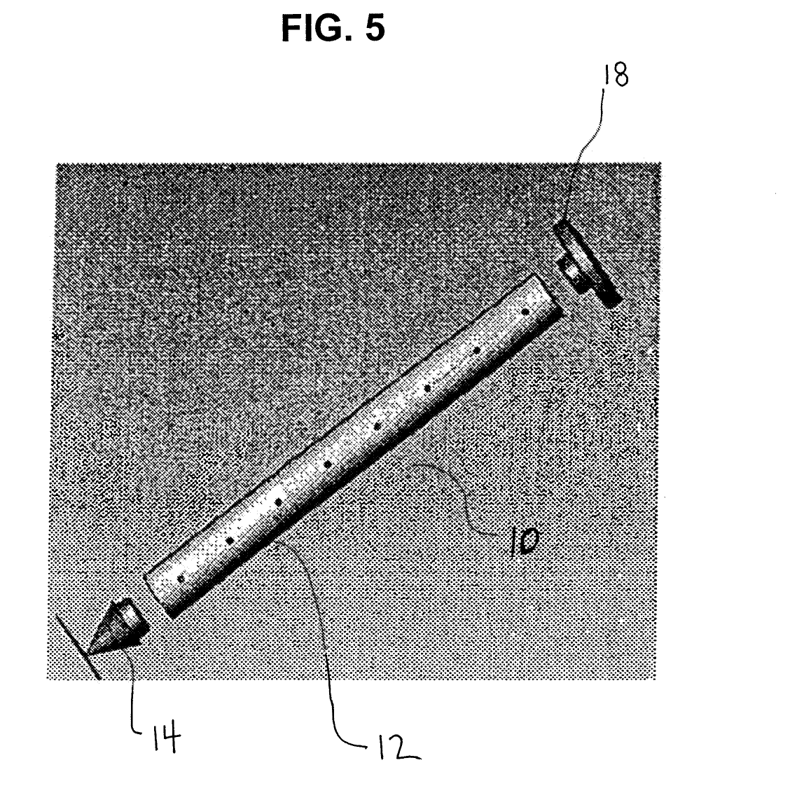

Drug delivery device 10 can be designed as a one, two or three piece set. In one embodiment, as shown in FIG. 5, drug delivery device 10 can be designed as a three piece set, e.g., as body member 12, end 14 and cap element 18, and assembled prior to use. For example, end 14 and cap element 18 can be removably attached to body member 12 by a friction fit or the outer side surface and inner side surface of end 14 and cap element 18 may be threaded to allow each of end 14 and cap element 18 to be screwed onto body member 12. Other engagement means are also envisioned such as pressed, locking or, in the absence of engagement means, sealed with an impermeable material.

A plurality of the drug delivery devices of the present invention can be used simultaneously or successively. Therefore, if a high concentration of the drug is needed for clinical treatment, a plurality of the devices can be used simultaneously, and if a releasing period of the drug should be extended, the devices can be used successively or additionally. Thus, even if a desired amount of the drug can be contained in a piece of the device, a desired amount of the drug can be released into the vitreous body by using the devices simultaneously or successively.

The dimensions of the drug delivery device of the present invention will depend on the intended application of the device, and will be readily apparent to those having ordinary skill in the art. By way of example, when the delivery device is used to deliver drugs to the posterior chamber of the eye, the device is preferably designed for insertion through a small incision that requires few to no sutures for scleral closure at the conclusion of the procedure. As such, the device is preferably inserted through an incision that is no more than about 1 mm in cross-section, e.g., ranging from about 0.25 mm to about 1 mm in diameter, more preferably less than about 0.5 mm in diameter. As such, the cross-section of the body member 12, is preferably no more than about 0.5 mm, and preferably ranging from about 0.4 mm to about 0.6 mm in internal diameter. If body member 12 is not cylindrical, the largest dimension of the cross section can be used to approximate the diameter for this purpose. When used to deliver drugs to the posterior chamber of the eye, body member 12 preferably has a length from its distal end 14 to its second end 16 that is less than about 1.5 cm, and preferably ranges from about 0.5 cm to about 1.5 cm such that when cap element 18 abuts the outer surface of the eye, the delivery mechanism is positioned near the posterior chamber of the eye. In general, the total length of member 12 will ordinarily not exceed about 1 cm, preferably not more than about 0.7 cm and most preferably not more than about 0.5 cm. and the delivery mechanism for delivering the drug to the area in need of treatment will be positioned at the pars plana region of the eye.

The drug delivery devices of the present invention may be used in a broad range of therapeutic applications. The drug delivery devices of the present invention are particularly useful in the treatment of an ophthalmic state, disease, disorder, injury or condition. Representative examples of such an ophthalmic state, disease, disorder, injury or condition include, but are not limited to, diabetic retinopathy, glaucoma, macular degeneration, retinitis pigmentosa, retinal tears or holes, retinal-detachment, retinal ischemia, acute retinopathies associated with trauma, inflammatory mediated degeneration, substantially linear shaped body member-surgical complications, damage associated with laser therapy including photodynamic therapy (PDT), surgical light induced iatrogenic retinopathy, drug-induced retinopathies, autosomal dominant optic atrophy, toxic/nutritional amblyopias; leber's hereditary optic neuropathy (LHOP), other mitochondrial diseases with ophthalmic manifestations or complications, angiogenesis; atypical RP; bardet-biedl syndrome; blue-cone monochromacy; cataracts; central areolar choroidal dystrophy; choroideremia; cone dystrophy; rod dystrophy; cone-rod dystrophy; rod-cone dystrophy; congenital stationary night blindness; cytomegalovirus retinitis; diabetic macular edema; dominant drusen; giant cell arteritis (GCA); goldmann-favre dystrophy; graves' ophthalmopathy; gyrate atrophy; hydroxychloroquine; iritis; juvenile retinoschisis; kearns-sayre syndrome; lawrence-moon bardet-biedl syndrome; leber congenital amaurosis; lupus-induced cotton wool spots; macular degeneration, dry form; macular degeneration, wet form; macular drusen; macular dystrophy; malattia leventinese; ocular histoplasmosis syndrome; oguchi disease; oxidative damage; proliferative vitreoretinopathy; refsum disease; retinitis punctata albescens; retinopathy of prematurity; rod monochromatism; RP and usher syndrome; scleritis; sector RP; sjogren-larsson syndrome; sorsby fundus dystrophy; stargardt disease and other retinal diseases.

In use, the drug delivery device is inserted into the eye to deliver the one or more pharmaceutically active agents. For example, in embodiments wherein the distal end 14 of substantially linear shaped body member 12 has a conical shape, device 10 is inserted into the eye by separating a portion of the conjunctival membrane of an eye from a portion of scleral tissue underlying the portion of the conjunctival membrane. An incision can be made through the portion of scleral tissue into the vitreous region of the eye such that an opening for insertion of the device is created. The device is inserted into the opening such that body member 12 of device 10 is situated in the vitreous region and cap element 18 abuts the outer surface of the eye. If desired, the portion of the conjunctival membrane can be sutured to the device. The device is maintained in the vitreous region until a predetermined dosage of the drug is delivered into the vitreous region. When finished, the device can be removed from the eye, and the portion of the conjunctival membrane is reattached over the opening in the portion of scleral tissue.

The present invention is not to be limited to ocular applications, and can also be useful in other limited access regions such as the inner ear.

The present invention also includes kits that contain one or more of the drug delivery devices of the present invention, preferably packaged in sterile condition. Kits of the invention also may include, for example, means for suturing or securing the device to the sclera, etc. for use with the device, preferably packaged in sterile condition, and/or written instructions for use of the device and other components of the kit.

The following examples are provided to enable one skilled in the art to practice the invention and are merely illustrative of the invention. The examples should not be read as limiting the scope of the invention as defined in the claims.

### EXAMPLE 1

The following materials were used in preparing a polymeric matrix for use in the drug delivery system of the present invention:
Diclofenamide (DCP), (Sigma D-32683)
PLGA (85:15), 0.53 dl/g IV, (Birmingham Polymers, Inc).

DCP and PLGA were mixed in a 35:65 w/w ratio and melt extruded using a Lab Mixing Extruder (LME), (Dynisco Instruments, Inc.). The ingredients were first allowed to mix inside the heated barrel for at least 5 minutes and then extruded by pulling filament strands of approximately 0.5 mm in diameter. The entire extruded batch was collected as strands and then physically mixed together and reextruded under the same process conditions. The final batch of filaments was collected and stored in a dry dessicator box FOR future use.

The process conditions used for the LME to prepare the 35% DCP implants were as follows:
Rotor Temperature: 125° C.
Header Temperature: 130° C.
Rotor RPM: 10 setting
Filament Line puller setting: 40-80

### EXAMPLE 2

A second polymeric matrix for use in the drug delivery system of the present invention was prepared in substantially the same manner as in Example 1. The following materials and process conditions were used for this example:

Materials:
35% Dichlorphenamide
10% (50:50) DL-PLGA, 0.39 I.V.
15% (75:25) DL-PLGA, 0.19 I.V.
35% DL-PLA, 0.24 I.V.
5% TPGS* *d-alpha tocopheryl polyethyleneglycol 1000 succinate

Process Conditions:
Rotor Temp: 90° C.
Header Temp: 95° C.
Rotor RPM: 30-40 setting
Filament Line puller setting: 55

### EXAMPLE 3

Preparation of a Non-Deformable Tack.

A precision bored hollow tube threaded on both ends made of 316L stainless steel was perforated with small—50 um holes using a laser. A threaded pointed member and threaded head piece were precision ground on a lathe using the same grade material as the hollow tube. The threaded pointed member was threaded onto the hollow tube.

### EXAMPLE 4

Preparation of a Drug Loaded Non-Deformable Tack.

The drug loaded filament containing degradable polymer matrix of Example 2 was cut to 5 mm length. The diameter of the implant was 0.4+0.02 mm. The cut filament was gently inserted into the hollow tube of Example 3 using a forcep with the pointed member already threaded on at the distal end. The tack was then fitted with the threaded head piece to complete the assembly of the tack device.

### EXAMPLE 5

Preparation of a Non-Deformable Tack.

A precision bored hollow tube (0.48 mm internal diameter (ID)) threaded on both ends made of 316L stainless steel was perforated in the tube wall with 8 holes (˜50 um) using a laser. A threaded pointed member and threaded head piece were precision ground on a lathe using the same grade material as the hollow tube. The threaded pointed member was threaded onto the hollow tube.

### EXAMPLE 6

Preparation of a Drug Loaded Non-Deformable Tack.

The drug loaded filament containing degradable polymer matrix of Example 2 was cut to 5 mm length. The diameter of the implant was 0.4+0.02 mm. The cut filament was gently inserted into the hollow tube of Example 5 using a forcep with the pointed member already threaded on at the distal end. The tack was then fitted with the threaded head piece to complete the assembly of the tack device.

### EXAMPLE 7

Preparation of a Non-Deformable Tack.

A precision bored hollow tube (0.48 mm ID) threaded on both ends made of 316L stainless steel was perforated in the tube wall with 32 holes (˜50 um) using a laser. A threaded pointed member and threaded head piece were precision ground on a lathe using the same grade material as the hollow tube. The threaded pointed member was threaded onto the hollow tube.

### EXAMPLE 8

Preparation of a Drug Loaded Non-Deformable Tack.

The drug loaded filament containing degradable polymer matrix of Example 2 was cut to 5 mm length. The diameter of the implant was 0.4+0.02 mm. The cut filament was gently inserted into the hollow tube of Example 7 using a forcep with the pointed member already threaded on at the distal end. The tack was then fitted with the threaded head piece to complete the assembly of the tack device.

### EXAMPLE 9

Testing of Drug Loaded Non-Deformable Tack.

The drug loaded non-deformable tacks of Examples 6 and 8 were each suspended in a release media in a vial from a rigid wire loop such that the tacks did not come in contact with the vial. The release media in the vial was 3 ml 2% fetal bovine serum (FBS)/phosphate buffer saline (PBS) which was removed at regular intervals—twice a week and every 3^{rd}/4^{th} day.

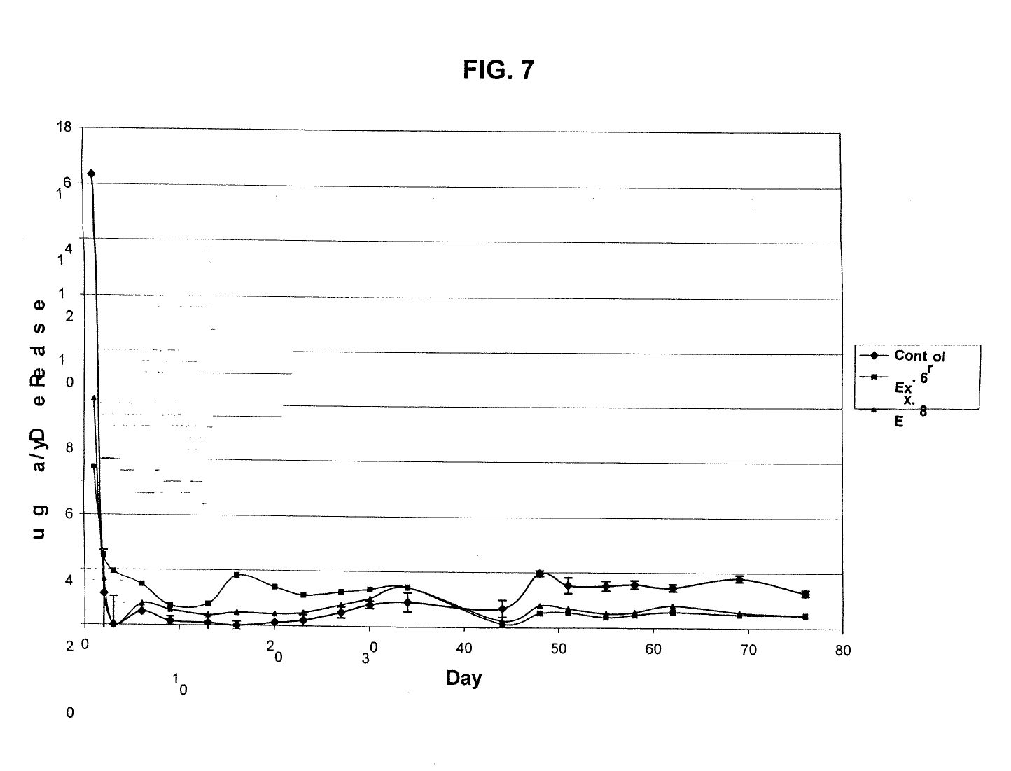

A control implant (DCP only) was similarly placed in the vial containing the same volume of release media and was allowed to freely move around in the vial. The entire set of vials—3 controls, 3 tacks with 8 holes and 3 tacks with 32 holes were placed on an orbital shaker unit and allowed to gently shake the contents of the vial. The shaker was itself placed inside an incubator that was maintained at 37° C. for the duration of the experiment. The sample media was prepped for HPLC analysis and the dichlorphenamide content was determined based on a standard analytical technique. The results of the tests are set forth in FIG. 7.

It will be understood that various modifications may be made to the embodiments disclosed herein. Therefore the above description should not be construed as limiting, but merely as exemplifications of preferred embodiments. For example, it will be manifest to those skilled in the art that various modifications may be made without departing from the spirit and scope of the underlying inventive concept. Other arrangements and methods may be implemented by those skilled in the art without departing from the scope and spirit of this invention. Moreover, those skilled in the art will envision other modifications within the scope and spirit of the features and advantages appended hereto.

What is claimed is:

1. An implantable drug delivery device for intraocular delivery comprising:
(a) a non-deformable, non-degradable, substantially linear shaped body member for housing a polymeric matrix comprising one or more pharmaceutically active agents and being implanted within a patient's eye during use of the device to deliver the one or more pharmaceutically active agents to the patient's eye;
(b) a delivery mechanism for delivery of the one or more pharmaceutically active agents; and
(c) a cap element that remains external to the eye and mates against the outer surface.

2. The implantable drug delivery device of claim 1, wherein the non-deformable, non-degradable, substantially linear shaped body member comprises a material having a Youngs Modulus of at least about 1 GPa.

3. The implantable drug delivery device of claim 1, wherein the non-deformable, non-degradable, substantially linear shaped body member comprises a material selected from steel, titanium, ceramic, ultra-high molecular weight polyethylene (UHMWPE), polymethylmethacrylate (PMMA) and polyether ether ketone (PEEK).

4. The implantable drug delivery device of claim 1, wherein the non-deformable, non-degradable, substantially linear shaped body member comprises stainless steel.

5. The implantable drug delivery device of claim 1, wherein the non-deformable, non-degradable, substantially linear shaped body member comprises a cobalt-chromium-molybdenum alloy.

6. The implantable drug delivery device of claim 1, wherein the polymeric matrix comprises a biodegradable or a non-biodegradable homo- or co-polymer.

7. The implantable drug delivery device of claim 6, wherein the polymeric matrix comprises a biodegradable homo- or co-polymer selected from the group consisting of poly(lactide)s, poly(glycolide)s, poly(lactide-co-glycolide)s, poly(lactic acid)s, poly(lactic acid-co-glycolic acid)s, polycaprolactones, polycarbonates, poly(ester amide)s, polyanhydrides, poly(amino acid)s, polyorthoesters, polyacetals, polycyanoacrylates, poly(ether ester)s, polydioxanones, poly(alkylene alkylate)s, copolymers of poly(ethylene glycol) and polyorthoesters, biodegradable polyurethanes and blends and copolymers thereof.

8. The implantable drug delivery device of claim 1, wherein the polymeric matrix comprises a poly(lactic acid-co-glycolic acid) copolymer.

9. The implantable drug delivery device of claim 1, wherein the one or more pharmaceutically active agents is selected from the group consisting of an anti-glaucoma agent, anti-cataract agent, anti-diabetic retinopathy agent, thiol cross-linking agent, anti-cancer agent, immune modulator agent, anti-clotting agent, anti-tissue damage agent, anti-inflammatory agent, anti-fibrous agent, non-steroidal anti-inflammatory agent, antibiotic, anti-pathogen agent, piperazine derivative, cycloplegic agent, miotic agent, mydriatic agent and mixtures thereof.

10. The implantable drug delivery device of claim 1, wherein the cap element mates the non-deformable, non-degradable, substantially linear shaped body member at a proximal end of the device.

11. The implantable drug delivery device of claim 1, wherein the non-deformable, non-degradable, substantially linear shaped body member has a conical shape at a distal end of the device.

12. The implantable drug delivery device of claim 1, wherein the delivery mechanism comprises one or more openings along the non-deformable, non-degradable, substantially linear shaped body member.

13. The implantable drug delivery device of claim 12, wherein the size and/or number of the one or more openings controls the rate of delivery of the one or more pharmaceutically active agents.

14. The implantable drug delivery device of claim 1, wherein the delivery mechanism comprises a permeable or semi-permeable material forming at least a portion of the non-deformable, non-degradable, substantially linear shaped body member.

15. A method of treating an ophthalmic state, disease, disorder, injury or condition, the method comprising:
(a) providing a drug delivery device comprising (i) a non-deformable, non-degradable, substantially linear shaped body member for housing a polymeric matrix comprising one or more pharmaceutically active agents to be delivered; (ii) a delivery mechanism for delivery of the one or more pharmaceutically active agents; and (iii) a cap element that remains external to the eye and mates against the outer surface of the patient's eye while the substantially linear shaped body member is inserted into the eye; and
(b) inserting the device into a patient's eye.

16. The method of claim 15, wherein the step of administering comprises:
creating an incision within an eye; and
inserting the drug delivery device through the incision until the cap element mates against the outer surface of the patient's eye.

17. The method of claim 15, wherein the non-deformable, non-degradable, substantially linear shaped body member comprises a material having a Youngs Modulus of at least about 1 GPa.

18. The method of claim 15, wherein the non-deformable, non-degradable, substantially linear shaped body member comprises a material selected from steel, titanium, ceramic, ultra-high molecular weight polyethylene (UHMWPE), polymethylmethacrylate (PMMA) and polyether ether ketone (PEEK).

19. The method of claim 15, wherein the polymeric matrix comprises a biodegradable or non-biodegradable homo- or co-polymer.

20. The method of claim 15, wherein the polymeric matrix comprises a biodegradable homo- or co-polymer selected from the group consisting of poly(lactide)s, poly(glycolide)s, poly(lactide-co-glycolide)s, poly(lactic acid)s, poly(lactic acid-co-glycolic acid)s, polycaprolactones, polycarbonates, poly(ester amide)s, polyanhydrides, poly(amino acid)s, polyorthoesters, polyacetals, polycyanoacrylates, poly(ether ester)s, polydioxanones, poly(alkylene alkylate)s, copolymers of poly(ethylene glycol) and polyorthoesters, biodegradable polyurethanes and blends and copolymers thereof.

21. The method of claim 15, wherein the polymeric matrix comprises a poly(lactic acid-co-glycolic acid) copolymer.

22. The method of claim 15, wherein the one or more pharmaceutically active agents is selected from the group consisting of an anti-glaucoma agent, anti-cataract agent, anti-diabetic retinopathy agent, thiol cross-linking agent, anti-cancer agent, immune modulator agent, anti-clotting agent, anti-tissue damage agent, anti-inflammatory agent, anti-fibrous agent, non-steroidal anti-inflammatory agent, antibiotic, anti-pathogen agent, piperazine derivative, cycloplegic agent, miotic agent, mydriatic agent and mixtures thereof.

23. The method of claim 15, wherein the non-deformable, non-degradable, substantially linear shaped body member of the device has a conical shape at a distal end of the device.

24. The method of claim 15, wherein the delivery mechanism comprises one or more openings along the non-deformable, non-degradable, substantially linear shaped body member.

25. The method of claim 15, wherein the size and/or number of the one or more openings controls the rate of delivery of the one or more pharmaceutically active agents.
